(19) United States
(12) Patent Application Publication
(10) Pub. No.: US 2004/0122477 A1
(43) Pub. Date: Jun. 24, 2004

(54) Fully implantable miniature neurostimulator for spinal nerve root stimulation as a therapy for angina and peripheral vascular disease

(76) Inventors: Todd K. Whitehurst, Santa Clarita, CA (US); James P. McGivern, Stevenson Ranch, CA (US)

Correspondence Address: ADVANCED BIONICS CORPORATION, 12740 SAN FERNANDO ROAD, SYLMAR, CA, 91342, US

(21) Appl. No.: 10/730,689

(22) Filed: Dec. 8, 2003

Related U.S. Application Data
(60) Provisional application No. 60/434,984, filed on Dec. 19, 2002.

(51) Int. Cl. A61N 1/18; A61N 1/36

(52) U.S. Cl. 607/9; 607/46

(57) ABSTRACT

Methods for treatment of peripheral vascular disease and angina include implantation of a miniature stimulator adjacent at least one tissue influencing blood circulation. Stimulation sites include the spinal cord dorsal columns and spinal roots. Stimulation parameters are tailored to increase coronary blood flow to treat angina, and/or to increase peripheral blood flow to treat PVD. In addition, the strength and/or duration of electrical stimulation required to produce a desired therapeutic effect may be determined based on a sensed response to and/or need for treatment. Thus, the stimulation parameters may be adjusted based on a sensed condition.

The present application claims the benefit of U.S. Provisional Patent Application Serial No. 60/434,984, filed Dec. 19, 2002, which application is incorporated herein by reference in its entirety.

### BACKGROUND OF THE INVENTION

Peripheral vascular disease (PVD), such as Raynaud's disease and thromboangiitis obliterans, affects blood vessels, especially of the extremities. PVD is a common circulation problem in which the arteries become narrowed or clogged. Thus, PVD is sometimes called peripheral arterial disease (PAD). The most common cause of PVD is atherosclerosis (often called “hardening of the arteries”). Atherosclerosis is the build up of a plaque of cholesterol and scar tissue that clogs the blood vessels.

PVD affects about one in twenty people over the age of 50, or 10 million people in the United States. More than half the people with PVD experience leg pain, numbness or other symptoms, but only about half of those with symptoms have been diagnosed with PVD and are seeing a doctor for treatment.

Angina is a disease marked by spasmodic attacks of intense suffocating pain. For instance, angina pectoris may be described as an aching, burning, or squeezing pain, or as a discomfort, heaviness, or pressure in the chest that occurs when an inadequate supply of blood reaches the heart muscle. Angina pectoris is usually felt in the chest, but may also be felt in the left shoulder, arms, neck, throat, jaw, or back. Angina pectoris is usually caused by a narrowing or blockage of the coronary arteries (blood vessels supplying blood to the heart), which is usually the result of atherosclerosis.

Estimates are that 6,600,000 people in the United States suffer from angina. The estimated age-adjusted prevalence of angina is greater in women than in men. Angina rates in women age 20 and older are 3.9 percent for non-Hispanic white women, 6.2 percent for non-Hispanic black women and 5.5 percent for Mexican-American women. Rates for men in these three groups are 2.6, 3.1 and 4.1 percent, respectively.

Existing treatments for PVD and angina suffer from a variety of disadvantages. Currently used medications tend to improve blood circulation (i.e., oxygen supply) only acutely, if at all. (Vasodilators can improve blood supply somewhat.) Existing surgical procedures are invasive, have high morbidity, and/or are often only temporarily beneficial. What is needed are less invasive systems and methods to effectively and efficiently deliver electrical stimulation to appropriate treatment sites to treat angina and PVD, and relieve patients of their symptoms.

### BRIEF SUMMARY OF THE INVENTION

The invention disclosed and claimed herein provides treatments for peripheral vascular disease (PVD) and angina and/or for relieving their symptoms using one or more implantable microstimulators for delivering electrical stimulation. The present invention overcomes the shortfalls of all prior art treatment devices by delivering such electrical stimulation to the spinal cord or spinal nerve root(s) via a miniature stimulator implanted entirely in the spinal column via a minimally invasive surgical procedure.

The stimulator used with the present invention possesses one or more of the following properties, among other properties:

at least one electrode for applying stimulating current to surrounding tissue;

electronic and/or mechanical components encapsulated in a hermetic package made from biocompatible material(s);

an electrical coil or other means of receiving energy and/or information inside the package, which receives power and/or data by inductive or radio-frequency (RF) coupling to a transmitting coil placed outside the body, thus avoiding the need for electrical leads to connect devices to a central implanted or external controller;

means for receiving and/or transmitting signals via telemetry;

means for receiving and/or storing electrical power within the stimulator; and

a form factor making the stimulator implantable via a minimally invasive procedure in a target area in the body.

A stimulator may operate independently, or in a coordinated manner with other implanted stimulators, other implanted devices, and/or with devices external to a patient's body. For instance, a stimulator may incorporate means of sensing a patient's condition, e.g., a means for sensing PVD. Sensed information may be used to control the electrical stimulation parameters in a closed loop manner. The sensing and stimulating means may be incorporated into a single stimulator, or a sensing means may communicate sensed information to at least one stimulator with stimulating means.

For most patients, a continuous or intermittent stimulation throughout the day is needed to provide an adequate amount of treatment. These patients may best utilize a stimulator that has a self-contained power source sufficient to deliver repeated pulses for at least several days and that can be recharged repeatedly, if necessary. In accordance with the teachings of the present invention, the use of a stimulator with a rechargeable battery thus provides these patients the portability needed to free the patient from reliance on RF power delivery. Alternatively, the power source may be a primary battery that may last several years.

For purposes of this patent application, it is sufficient to note that RF controlled stimulators receive power and control signals from an extra corporeal antenna coil via inductive coupling of a modulated RF field. Battery-operated stimulators incorporate a power source within the device itself but rely on RF control, inductive linking, or the like to program stimulus sequences and, if a rechargeable/replenishable power source is used, to recharge/replenish the power source, when needed. In accordance with the present invention, each implanted stimulator may be commanded to produce an electrical pulse of a prescribed magnitude and duration and at a repetition rate sufficient to treat the targeted tissue.

For instance, stimulation may be initiated by start and stop commands from a patient-governed control switch or controller, which may be handheld, containing a microprocessor and appropriate nonvolatile memory, such as electronically erasable programmable read-only-memory (EEPROM). The controller may control the implantable stimulator by any of various means. For instance, the stimulator may sense the proximity of a permanent magnet located in the controller, or may sense RF transmissions from the controller.

### BRIEF DESCRIPTION OF THE DRAWINGS

The above and other aspects of the present invention will be more apparent from the following more particular description thereof, presented in conjunction with the following drawings wherein:

FIG. 1 illustrates the relation of spinal nerve roots to vertebrae;

FIG. 2A depicts nerve pathways in and near the thoracic spinal cord;

FIG. 2B illustrates the principal fiber tracts of the spinal cord;

FIG. 3 depicts a section through a vertebra;

FIG. 4 illustrates an exemplary embodiment of a stimulation system of the present invention;

FIG. 5 illustrates exemplary external components of the invention; and

FIG. 6 depicts a system of implantable devices that communicate with each other and/or with external control/programming devices.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

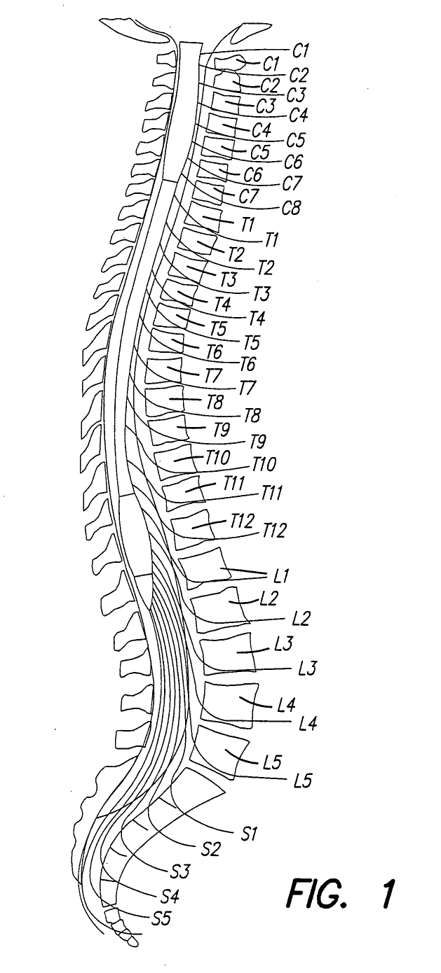

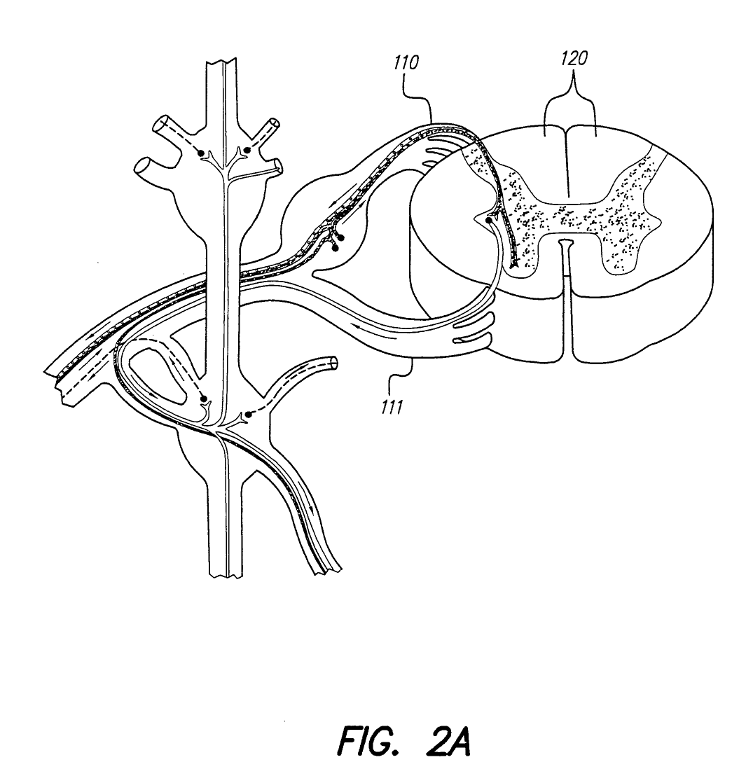

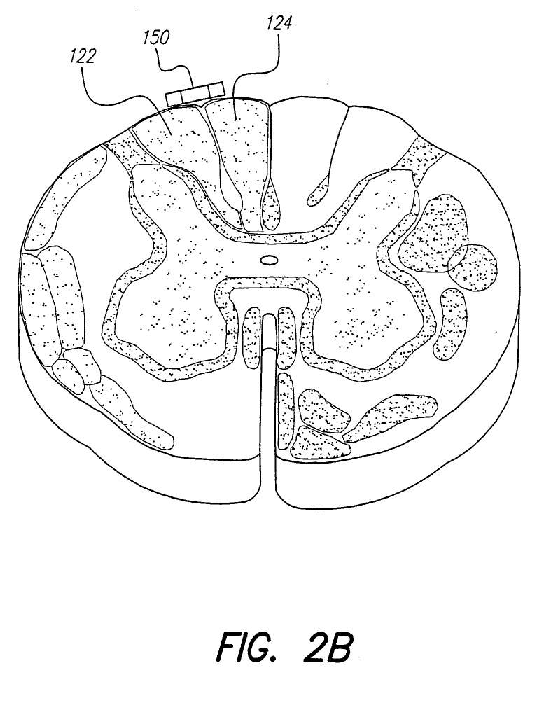

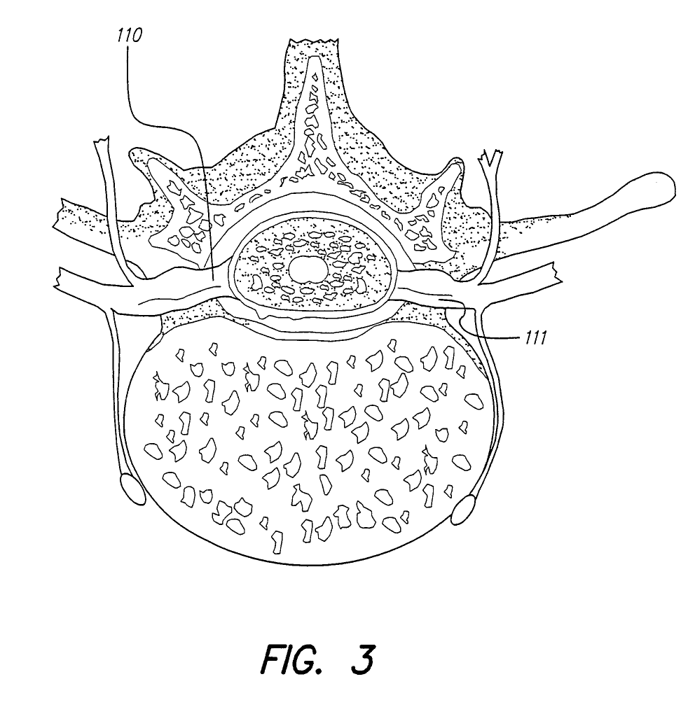

As stated above, the relation of spinal nerve roots to vertebrae is illustrated in FIG. 1. FIG. 2A depicts nerve pathways in and near the thoracic part of the spinal cord. FIG. 2B illustrates the principal fiber tracts of the spinal cord, and FIG. 3 depicts a section through a vertebra.

Spinal Cord Stimulation (SCS) for Angina Pectoris and Peripheral Vascular Disease (PVD)

The gate theory of pain proposed by Meizack and Wall in 1965 [see Meizack R, Wall P D. “Pain mechanisms: a new theory.” Science 1965; 150:971-9] led to the first spinal cord stimulator being implanted by Norman Shealy in 1967 for cancer pain. Use of SCS in angina was reported in 1984 as a chance finding in a patient who had a stimulator for another reason. [See Sandric, et al. “Clinical and electrocardiographic improvement of ischemic heart disease after spinal cord stimulation.” Acta Neurochir Suppl 1984; 33:543-6.] SCS systems were first specifically implanted for intractable angina in Australia in 1987. Since then, there have been over 70 publications on SCS in refractory angina. These studies have confirmed improvement in quality of life of these patients, fewer ischemic episodes, and reduced frequency of hospital admissions. Moreover, these effects are long-lasting and are obtained at negligible risk.

Clinicians are generally concerned about the potential risks of masking myocardial ischemia with SCS. Studies have demonstrated that SCS decreases lactate production with pacing and total ischemic burden, without an increase in silent ischemia. In a study of fifty patients with coronary artery disease and severe intractable angina treated with SCS for 1-57 months, Andersen, et al. found that SCS does not mask the pain of an acute Ml. [See Andersen, et al. “Does pain relief with spinal cord stimulation for angina conceal myocardial infarction?” British Heart Journal 1994; 71:419-421.] It has also been found that mortality rates in patients with SCS systems are similar to those of the general population of patients with coronary artery disease.

SCS has been demonstrated to promote local blood flow and ischemic ulcer healing in patients with peripheral vascular disease. Positron emission tomography (PET) has shown a more homogenous pattern of coronary flow following SCS in patients with myocardial ischemia but no increase in total flow. This redistribution of flow to areas that were previously ischemic may explain the increase in exercise capacity prior to the inevitable onset of angina. To date, there has been no proof of an increase in coronary flow velocity when patients undergo pacing stress with SCS. [See Norrsell, et al. “Effects of spinal cord stimulation on coronary blood flow velocity.” Coronary Artery Disease 1998; 9:273-8.]

It has been proposed that SCS may alter sympathetic/parasympathetic balance, but no change in heart rate variability has been shown in a group of post-SCS patients. [See Hautvast, et al. “Effect of spinal cord stimulation on heart rate variability and myocardial ischemia in patients with chronic intractable angina pectoris—a prospective ambulatory electrocardiographic study.” Clinical Cardiology 1998; 21:33-8.] However, a decrease in resting heart rate and features suggestive of a functional sympathectomy were found in 25 SCS patients without coronary disease. [See Meglio, et al. “Spinal cord stimulation affects the central mechanisms of regulation of heart rate.” Applied Neurophysiology 1986; 49:139-146.] Cerebral PET scanning of patients with an SCS system demonstrated changes in blood flow in areas that are known to be related to pain perception in angina. [See Hautvast, et al. “Relative changes in regional cerebral blood flow during spinal cord stimulation in patients with refractory angina pectoris.” European Journal of Neuroscience 1997; 9:1178-83, and Rosen, et al. “Central nervous pathways mediating angina pectoris.” Lancet 1994; 344:147-150.]

SCS improves microcirculatory blood flow, relieves diabetic neuropathic and ischemic pain and reduces the amputation rate in patients with severe peripheral arterial occlusive disease [Huber, 1996; Petrakis, 2000]. In order to evaluate whether transcutaneous oxygen tension (TcPO₂) measurements can be used as a specific prognostic parameter in the assessment of suitability for permanent device implantation in a prospective controlled study on diabetic patients with peripheral arterial occlusive disease, [Petrakis, 2000] implanted 60 patients (39 men, 21 women; mean age: 60 years; range: 46-75) with an SCS system for severe peripheral vascular disease, after failed conservative or surgical treatment. The primary pathology was diabetic vascular disease. Pedal TcPO₂ was assessed on the dorsum of the foot, and ankle, and toe pressure Doppler measurements were performed before, two weeks, and four weeks after implantation.

Pain relief of over 75% and limb salvage were achieved in 35 diabetic patients, while in 12 a partial success with pain relief over 50% and limb salvage for at least 6 months were obtained. In 13 patients the method failed and the affected limbs were amputated. Clinical improvement and SCS success were associated with increases of TcPO₂ within the first two weeks after implantation (temporary period). Limb salvage was achieved with significant increase of TcPO₂ within the first two weeks of the testing period unrelated to the stage of the disease and the initial TcPO₂ value. TcPO₂ changes were related to the presence of adequate paresthesias and warmth in the painful area during the trial period. The systolic ankle/brachial blood pressure index (ABI) and toe pressure did not change under stimulation. [Petrakis, 2000] concluded that a two-week testing period should be performed in all diabetic patients treated with spinal cord stimulation for peripheral arterial occlusive disease to identify the candidates for permanent implantation. Only diabetic patients with significant increases of TcPO₂ and clinical improvement during the test period should be considered for permanent implantation and not merely all patients with pain relief.

SCS versus Coronary Artery Bypass Graft (CABG) Surgery for Angina Pectoris

In 1998, Mannheimer, et al. compared SCS to coronary artery bypass graft (CABG) surgery in 104 high-risk patients who were undergoing intervention for symptomatic reasons only and who had an expected increased risk of surgical complications. [See Mannheimer, et al. “Electrical stimulation versus coronary artery bypass surgery in severe angina pectoris. The ESBY study.” Circulation 1998; 97:1157-63.] The patients were assessed with respect to symptoms, exercise capacity, ischemic ECG changes during exercise, rate-pressure product, mortality, and cardiovascular morbidity before and six months after the operation. The study found that both groups had approximately the same significant decrease in frequency of angina attacks as well as approximately the same significant decrease in the use of short-acting nitrates. The primary aim of both treatments is to improve quality of life by reducing symptoms. In this regard, both SCS and CABG produced similar benefits. CABG produced an additional improvement in ischemia on exercise testing at six months. Eight total deaths occurred during the follow-up period: seven total in the CABG group (four perioperative) and one in the SCS group. Cerebrovascular morbidity was also lower in the SCS group. (Most patients lose approximately ten IQ points as a result of CABG surgery.)

In a retrospective analysis of 19 patients implanted with SCS systems between 1987 and 1997, Murray, et al. found that the annual admission rate after CABG surgery was 0.97 per patient per year, compared with 0.27 after SCS. [See Murray, et al. “Spinal cord stimulation significantly decreases the need for acute hospital admission for chest pain in patients with refractory angina pectoris.” Heart 1999 July; 82(1):89-92.] The mean hospital time per patient per year after CABG was 8.3 days versus 2.5 days after SCS. No unexplained ECG changes were observed during follow-up, and SCS patients presented with unstable angina and acute Ml in the usual way. The study concludes that SCS effectively prevents hospital admissions in patients with refractory angina without masking serious ischemic symptoms or leading to silent infarction.

SCS Electrode Location and Stimulation Parameters

The electrodes for SCS for angina pectoris are typically implanted in the epidural space of the low cervical and high thoracic spinal segments, i.e., C7, T1, and T2. The stimulation voltage employed ranges from 0.7 to 9.5 volts (mean 4.2-4.5 volts), with an impedance range from 560 to 1667 Ω (mean 821-920 Ω). The stimulation frequency is typically set to 85 pps, although some studies have used frequencies as low as 20 pps with some efficacy. The pulse width typically used is 210 μsec. Intermittent stimulation is generally used. Typically the device is activated episodically by the patient, in response to anginal pain; studies have found the device active only 10-15% of a given week. [See, e.g., DeJongste, et al. “Stimulation characteristics, complications, and efficacy of spinal cord stimulation systems in patients with refractory angina: a prospective feasibility study.” Pacing and Clinical Electrophysiology 1994 November; 17(11 Pt 1):1751-60, and Jessurun, et al. “Longevity and costs of spinal cord stimulation systems in patients with refractory angina pectoris.” Third Annual Symposium on Pacing Leads, Ferrara, Italy, Sep. 11-13, 1997.]

Transmyocardial Revascu Larization Surgery

Transmyocardial revascularization (TMR) is a procedure designed to relieve severe angina in patients who are not candidates for bypass surgery or angioplasty. During TMR, a surgeon uses a laser to drill a series of holes from the outside of the heart into the heart's pumping chamber. Twenty to forty 1 mm laser channels are created during the procedure. Bleeding from the channels stops after a few minutes of pressure from the surgeon's finger. In some patients TMR is combined with bypass surgery. How TMR reduces angina still isn't fully understood. The laser may stimulate new blood vessels to grow (angiogenesis). It may destroy nerve fibers to the heart, making patients unable to feel their chest pain. In some cases, the channels may remain open, which would let oxygen-rich blood from the pumping chamber flow into the channel and then into the heart muscle.

TMR is FDA approved for use in patients with severe angina who have no other treatment options. It has also produced early promising results in three large multi-center clinical trials. The angina of 80-90 percent of patients who had this procedure has significantly improved (at least 50 percent) through one year after surgery. There's still limited follow-up data as to how long this procedure might last, however.

Limitations of traditional SCS systems include the bulky implantable pulse generator (IPG), limited life of an IPG with a primary battery, and the inconvenience of an RF powered system, among other limitations. Also, the procedure for implanting a traditional SCS system involves major surgery, with multiple incisions, local and general anesthetic, the risks of infection and other complications, and lengthy recovery time associated therewith.

This invention provides a means for chronically stimulating the spinal cord or spinal nerve root(s) with a miniature implantable neurostimulator that can be implanted with a minimal surgical procedure. According to the present invention, a miniature implantable neurostimulator, such as a bionic neuron (i.e., BION®) may be implanted via a minimal surgical procedure (e.g., via a small incision and through a cannula, endoscopically, laparoscopically) in the spinal cord or in the spinal column adjacent to a spinal nerve root to treat angina and/or PVD and/or the symptoms thereof. For example, the spinal nerve roots 110 and 111 lie within the spinal column, and a miniature implantable neurostimulator may be placed in the spinal column for stimulation of spinal root(s) 110 and/or 111. A more complicated surgical procedure, such as a laminectomy, may be required for sufficient access to a targeted nerve fiber(s), or for fixing the neurostimulator in place.

As indicated above, the present invention is directed to treating PVD and angina using one or more small, implantable neurostimulators, referred to herein as “microstimulators”. The microstimulators of the present invention are preferably similar to or of the type referred to as Bionic Neuron (also referred to as a BION® microstimulator) devices. The following documents describe various details associated with the manufacture, operation, and use of BION implantable microstimulators, and are all incorporated herein by reference:

| Application/ | | |
|---|---|---|
| Patent/ | Filing/Publi- | |
| Publication No. | cation Date | Title |
| U.S. Pat. No. | Issued | Implantable Microstimulator |
| 5,193,539 | Mar. 16, 1993 | |
| U.S. Pat. No. | Issued | Structure and Method of |
| 5,193,540 | Mar. 16, 1993 | Manufacture of an Implant- |
| | | able Microstimulator |
| U.S. Pat. No. | Issued | Implantable Device Having |
| 5,312,439 | May 17, 1994 | an Electrolytic Storage |
| | | Electrode |
| PCT Publication | published | Battery-Powered Patient |
| WO 98/37926 | Sep. 3, 1998 | Implantable Device |
| PCT Publication | published | System of Implantable De- |
| WO 98/43700 | Oct. 8, 1998 | vices For Monitoring and/or |
| | | Affecting Body Parameters |
| PCT Publication | published | System of Implantable De- |
| WO 98/43701 | Oct. 8, 1998 | vices For Monitoring and/or |
| | | Affecting Body Parameters |
| U.S. Pat. No. | Issued | Improved Implantable Micro- |
| 6,051,017 | Apr. 18, 2000 | stimulator and Systems |
| | | Employing Same |
| | published | Micromodular Implants to |
| | September 1997 | Provide Electrical Stimu- |
| | | lation of Paralyzed Muscles |
| | | and Limbs, by Cameron, et |
| | | al., published in IEEE Trans- |
| | | actions on Biomedical Engi- |
| | | neering, Vol. 44, No. 9, |
| | | pages 781-790. |

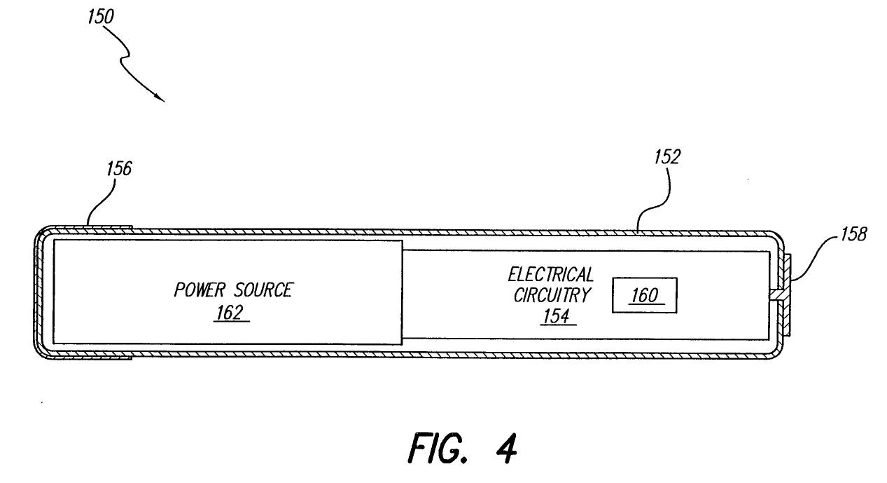

To treat angina pectoris, for example, a microminiature stimulator 150, such as a BION microstimulator, illustrated, e.g., in FIGS. 2B and 4, may be implanted adjacent to the cervical and/or thoracic spinal cord dorsal column(s). As another example, to treat PVD of the lower limb(s), a microstimulator 150 may be implanted adjacent the lumbar and/or sacral spinal cord dorsal column(s).

Based on the gate control theory described earlier, stimulating fast-conducting, larger diameter nerve fibers will block, or gate, the slower pain signals from reaching the brain. The somatic sensory fibers responsible for touch, pressure, and position sense are carried through relatively large diameter nerve fibers (i.e., A-α and/or A-β fibers), while smaller diameter nerve fibers (e.g., A-δ and/or C fibers) carry pain signals. As such, angina and/or PVD may be treated with stimulation additionally or alternatively applied to the larger diameter nerve fibers, which larger diameter fibers have a relatively lower threshold of activation than smaller diameter fibers. Excitatory stimulation of relatively low frequency (e.g., less than about 50-100 Hz) and/or relatively low amplitude (e.g., less than about 15 mA) stimulation is likely to lead to the activation of these relatively large diameter non-nociceptive sensory fibers.

In accordance with the present invention, a single microstimulator 150 may be implanted, or two or more microstimulators may be implanted to achieve greater stimulation of the targeted tissue, or for a longer period of time. In the example of FIG. 4, microstimulator device 150 includes a narrow, elongated case 152 containing electronic circuitry 154 connected to electrodes 156 and 158, which may pass through the walls of the case at either end. Alternatively, electrodes 156 and/or 158 may be built into and/or onto the case and/or arranged on a distal portion of a lead, as described below. As detailed in the referenced publications, electrodes 156 and 158 generally comprise a stimulating electrode (to be placed close to the nerve) and an indifferent electrode (for completing the circuit). Other configurations of microstimulator device 150 are possible, as is evident from the above-referenced publications.

A preferred implantable microstimulator 150 is sufficiently small to permit its placement near the structures to be stimulated. (As used herein, “adjacent” and “near” mean as close as reasonably possible to target tissue(s), including touching or even being positioned within the tissue, but in general, may be as far as can be reached with the stimulation pulses.) As such, case 152 may have a diameter of about 4-5 mm, or only about 3 mm, or even less than about 3 mm. In these configurations, case length may be about 25-35 mm, or only about 20-25 mm, or even less than about 20 mm. The shape of the microstimulator may be determined by the structure of the desired target, the surrounding area, and the method of implantation. A thin, elongated cylinder with electrodes at the ends, as shown in FIG. 4, is one possible configuration, but other shapes, such as rounded cylinders, spheres, disks, and helical structures, are possible, as are different configurations of and/or additional electrodes.

Microstimulator 150 is preferably implanted with a surgical insertion tool specially designed for the purpose (see, e.g., U.S. Pat. No. 6,582,441), or may be placed, for instance, via a small incision and through a small cannula. Alternatively, device 150 may be implanted via conventional surgical methods, or may be inserted using other endoscopic or laparoscopic techniques. A more complicated surgical procedure may be required for purposes of fixing the microstimulator in place.

The external surfaces of stimulator 150 are advantageously composed of biocompatible materials. To protect the electrical components inside stimulator 150, at least a portion of case 152 is hermetically sealed. For instance, stimulator case 152 may be made of, for instance, glass, ceramic, or other material that provides a hermetic package that excludes water vapor but permits passage of electromagnetic fields used to transmit data and/or power. For additional protection against, e.g., impact, the case may be made of metal (e.g., titanium) or ceramic, which materials are also, advantageously, biocompatible. In addition, stimulator 150 may be configured to be Magnetic Resonance Imaging (MRI) compatible. Electrodes 156 and 158 may be made of a noble or refractory metal or compound, such as platinum, iridium, tantalum, titanium, titanium nitride, niobium, or alloys of any of these, in order to avoid corrosion or electrolysis, which could damage the surrounding tissues and the device.

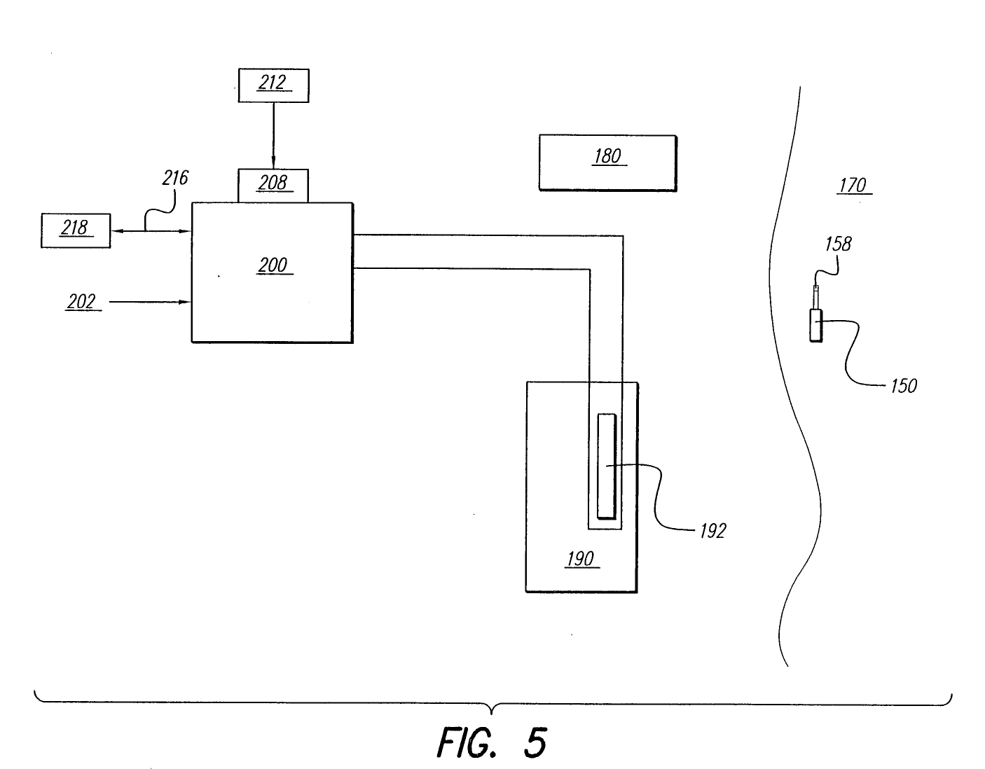

In some embodiments of the instant invention, microstimulator 150 comprises at least one, leadless electrode. However, one, some, or all electrodes may alternatively be located at the end of short, flexible leads (e.g., see FIG. 5) as described in U.S. patent application Ser. No. 09/624,130, filed Jul. 24, 2000 (which claims priority to U.S. Provisional Patent Application No. 60/156,980, filed Oct. 1, 1999), which is incorporated herein by reference in its entirety. Other configurations may also permit electrical stimulation to be directed more locally to specific tissue a short distance from the surgical fixation of the bulk of the implantable stimulator 150, while allowing elements of stimulator 150 to be located in a more surgically convenient site. Such configurations minimize the distance traversed and the surgical planes crossed by the device and any lead(s). In most embodiments, the leads are no longer than about 150 mm.

Microstimulator 150 contains, when necessary and/or desired, electronic circuitry 154 (FIG. 4) for receiving data and/or power from outside the body by inductive, radio-frequency (RF), or other electromagnetic coupling. In some embodiments, electronic circuitry 154 includes an inductive coil for receiving and transmitting RF data and/or power, an integrated circuit (IC) chip for decoding and storing stimulation parameters and generating stimulation pulses (either intermittent or continuous), and additional discrete components required to complete the circuit functions, e.g. capacitor(s), resistor(s), coil(s), and the like. Circuitry 154 may dictate, for instance, the amplitude and duration of the electrical current pulses.

Microstimulator 150 also includes, when necessary and/or desired, a programmable memory 160 for storing set(s) of data, stimulation, and/or control parameters. Among other things, memory 164 may allows stimulation and/or control parameters to be adjusted to settings that are safe and efficacious with minimal discomfort for each individual. Specific parameters may provide therapeutic advantages for different patients or for various types and classes of angina and/or PVD. For instance, some patients may respond favorably to intermittent stimulation, while others may require continuous stimulation for treatment and relief.

In addition, different parameters may have different effects on different tissue. Therefore, stimulation and control parameters may be chosen to target specific neural or other cell populations and/or to exclude others, or to increase activity in specific neural or other cell populations and/or to decrease activity in others. For example, relatively low frequency neurostimulation (i.e., less than about 50-100 Hz) may have an excitatory effect on surrounding neural tissue, leading to increased neural activity (“excitatory stimulation”), whereas relatively high frequency neurostimulation (i.e., greater than about 50-100 Hz) may have an inhibitory effect, leading to decreased neural activity (“inhibitory stimulation”). As another example, relatively low levels of stimulation current (typically less than about 15 mA, but dependent on the distance between electrodes and nerve fibers) are likely to recruit only relatively large diameter fibers (e.g., A-α and/or A-β fibers), while nociceptive fibers are typically relatively small diameter fibers (e.g., A-δ and/or C fibers).

Some embodiments of implantable stimulator 150 also includes a power source and/or power storage device 162 (FIG. 4). Possible power options, described in more detail below, include but are not limited to an external power source coupled to stimulator 150 (e.g., via an RF link), a self-contained power source utilizing any suitable means of generation or storage of energy (e.g., a primary battery, a replenishable or rechargeable battery such as a lithium ion battery, an electrolytic capacitor, a super- or ultra-capacitor, or the like), and if the self-contained power source is replenishable or rechargeable, means of replenishing or recharging the power source (e.g., an RF link, an optical link, a thermal link, or other energy-coupling link).

According to certain embodiments of the invention, a microstimulator operates independently. According to other embodiments of the invention, a microstimulator operates in a coordinated manner with other microstimulator(s), other implanted device(s), and/or other device(s) external to the patient's body. For instance, a microstimulator may control or operate under the control of another implanted microstimulator(s), other implanted device(s), or other device(s) external to the patient's body. A microstimulator may communicate with other implanted microstimulators, other implanted devices, and/or devices external to a patient's body via, e.g., an RF link, an ultrasonic link, a thermal link, or an optical link. Specifically, a microstimulator may communicate with an external remote control (e.g., patient and/or physician programmer) that is capable of sending commands and/or data to a microstimulator and that is preferably capable of receiving commands and/or data from a microstimulator.

In certain embodiments, and as illustrated in FIG. 5, the patient 170 switches stimulator 150 on and off by use of controller 180, which may be handheld. Implantable stimulator 150 may be operated by controller 180 by any of other various means, including sensing the proximity of a permanent magnet located in controller 180, sensing RF transmissions from controller 180, or the like.

Additional and alternative exemplary external components for programming and/or providing power to various embodiments of stimulator 150 are also illustrated in FIG. 5. When communication with such a stimulator 150 is desired, patient 170 is positioned on or near external appliance 190, which appliance contains one or more inductive coils 192 or other means of communication (e.g., RF transmitter and receiver). External appliance 190 is connected to or is a part of external circuitry appliance 200 which may receive power 202 from a conventional power source. External appliance 200 contains manual input means 208, e.g., a keypad, whereby the patient 170 or a caregiver 212 (e.g., a clinician) may request changes in stimulation parameters produced during the normal operation of the implantable stimulator 150. In these embodiments, manual input means 208 preferably includes various electro-mechanical switches and/or visual display devices that provide the patient and/or caregiver with information about the status and prior programming of the implantable stimulator 150.

Alternatively or additionally, external electronic appliance 200 is provided with an electronic interface means 216 for interacting with other computing means 218, such as via serial interface cable or infrared link to a personal computer or telephone modem or the like. Such interface means 216 may permit a clinician to monitor the status of the implant and prescribe new stimulation parameters from a remote location.

One or more of the external appliance(s) may be embedded in a cushion, mattress cover, garment, or the like. Other possibilities exist, including a strap, patch, or other structure(s) that may be affixed to the patient's body or clothing. External appliances may include a package that can be, e.g., worn on the belt, may include an extension to a transmission coil affixed, e.g., with a Velcro® band or an adhesive, or may be combinations of these or other structures able to perform the functions described herein.

In order to help determine the strength and/or duration of electrical stimulation required to produce the desired therapeutic effect, in some embodiments, a patient's response to and/or need for treatment is sensed, e.g., via ECG changes, an oxygen sensor or a flowmeter in the coronary circulation to detect indicators of angina. As another example, a microstimulator may incorporate means for sensing indicators of PVD, such as via an oxygen sensor or flowmeter in a limb. Sensed information may be used to control the stimulation parameters of a microstimulator in a closed-loop manner. According to some embodiments of the invention, the sensing and stimulating means are both incorporated into a single microstimulator. Thus, when microstimulator 150 is implanted, for example, near dorsal column 120, signals from a sensor built into microstimulator 150 may be used to adjust stimulation parameters. For instance, with stimulator 150 near dorsal column 120, stimulation may be initiated or amplitude increased if increased dorsal column activity is sensed via ENG. In another example, with stimulator 150 implanted in the spinal column near dorsal root 100, stimulation may be initiated or amplitude increased if ECG changes suggestive of angina are sensed.

According to other embodiments, the sensing means are incorporated into at least one “microstimulator” (that may or may not have stimulating means), and the sensed information is communicated to at least one other microstimulator with stimulating means. A microstimulator or other sensor may additionally or alternatively incorporate means of sensing other measures of the state of the patient, e.g., EMG, acceleration, patient activity, respiratory rate, medication levels, neurotransmitter levels, hormone levels, interleukin levels, cytokine levels, lymphokine levels, chemokine levels, growth factor levels, enzyme levels, and/or levels of other blood-borne compounds. For instance, one or more Chemically Sensitive Field-Effect Transistors (CHEMFETs), such as Enzyme-Selective Field-Effect Transistors (ENFETs) or Ion-Sensitive Field-Effect Transistors (ISFETs, as are available from Sentron CMT of Enschede, The Netherlands), may be used.

Thus, a “microstimulator” dedicated to sensory processes may communicate with a microstimulator that provides the stimulation pulses. For instance, a microstimulator, such as a BION® manufactured by Advanced Bionics of Sylmar, Calif., may be used to detect abnormal cardiac electrocardiogram (ECG) events. A BION may use one of many algorithms for analyzing ECGs. These algorithms can operating in the frequency domain, time domain or both. They may employ linear, non-linear, or statistical analysis to categorize the electrogram as originating from various modes, i.e., normal sinus rhythms, sinus tachycardia, ventricular tachycardia, and ventricular fibrillation. In addition, by finding the P, R, and T waves or analyzing the timing of the relationships and durations of the P-wave, QRS complex, and T-wave, it is possible to identify various disease states and make predictive diagnosis about perfusion of the myocardium. Other abnormalities that may be monitored include ST segment elevation, T wave peaking or inversion, among others discussed earlier. See, for instance, U.S. Pat. No. 5,513,644, titled “Cardiac arrhythmia detection system for an implantable stimulation device,” which is incorporated herein by reference in its entirety.

Other methods of determining the required stimulation include an oxygen or flow sensor in the coronary circulation and/or in a limb, as well as other methods mentioned herein, and yet others that will be evident to those of skill in the art upon review of the present disclosure. The sensed information may be used to control the electrical and/or control parameters in a closed-loop manner.

For instance, in several embodiments of the present invention, a first and second “stimulator” are provided. The second “stimulator” periodically (e.g. once per minute) records e.g., ECG, which it transmits to the first stimulator. Implant circuitry 154 may, if necessary, amplify, filter, process, then transmit these sensed signals, which may be analog or digital. The first stimulator uses the sensed information to adjust stimulation parameters according to an algorithm programmed, e.g., by a physician. For example, amplitude of stimulation may be initiated or increased in response to ST segment elevation and/or T wave inversion. More preferably, one “microstimulator” performs the sensing, stimulation parameter adjustments, and current generating functions.

While a microstimulator may also incorporate means of sensing angina, PVD, or their symptoms, it may alternatively or additionally be desirable to use a separate or specialized implantable device to sense and telemeter physiological conditions/responses in order to adjust stimulation parameters. This information may then be transmitted to an external device, such as external appliance 220, or may be transmitted directly to implanted stimulator(s) 150. However, in some cases, it may not be necessary or desired to include a sensing function or device, in which case stimulation parameters are determined and refined, for instance, by patient feedback.

Thus, it is seen that in accordance with the present invention, one or more external appliances are preferably provided to interact with microstimulator 150 to accomplish one or more of the following functions:

Function 1: If necessary, transmit electrical power from the external electronic appliance 200 via appliance 190 to the implantable stimulator 150 in order to power the device and/or recharge the power source/storage device 162. External electronic appliance 200 may include an automatic algorithm that adjusts stimulation parameters automatically whenever the implantable stimulator(s) 150 is/are recharged.

Function 2: Transmit data from the external appliance 200 via the external appliance 190 to the implantable stimulator 150 in order to change the operational parameters (e.g., electrical stimulation parameters) used by stimulator 150.

Function 3: Transmit sensed data indicating a need for treatment or in response to stimulation (e.g., ECG) from implantable stimulator 150 to external appliance 200 via external appliance 190.

Function 4: Transmit data indicating state of the implantable stimulator 150 (e.g., battery level, stimulation settings, etc.) to external appliance 200 via external appliance 190.

By way of example, a treatment modality for PVD may be carried out according to the following sequence of procedures:

1. A stimulator 150 is implanted so its electrodes 156 and/or 158 are adjacent to a lumbar dorsal column 120.

2. Using Function 2 described above (i.e., transmitting data) of external electronic appliance 200 and external appliance 190, implantable stimulator 150 is commanded to produce a series of excitatory electrical stimulation pulses.

3. Set stimulator on/off period to an appropriate setting, e.g., continuously on.

4. After each stimulation pulse, series of pulses, or at some other predefined interval, any change in dorsal column activity is sensed (via ENG), preferably by one or more electrodes 156 and 158 of implantable stimulator 150. These responses are converted to data and telemetered out to external electronic appliance 200 via Function 3.

5. From the response data received at external appliance 200 from the implantable stimulator 150, or from other assessment, the stimulus threshold for obtaining a reflex response is determined and is used by a clinician acting directly 212 or by other computing means 218 to transmit the desired stimulation parameters to the implantable stimulator 150 in accordance with Function 2. Alternatively, external appliance 200 makes the proper adjustments automatically, and transmits the proper stimulation parameters to stimulator 150. In yet another alternative, stimulator 150 adjusts stimulation parameters automatically based on the sensed response.

6. When patient 170 desires to invoke an electrical stimulation to alleviate symptoms (e.g., pain, loss of function, etc.), patient 170 employs handheld controller 180 to set the implantable stimulator 150 in a state where it delivers a prescribed stimulation pattern from a predetermined range of allowable stimulation patterns.

7. Patient 170 employs controller 180 to turn off stimulator 150, if desired.

8. Periodically, the patient or caregiver recharges the power source/storage device 162 of implantable stimulator 150, if necessary, in accordance with Function 1 described above (i.e., transmit electrical power).

In another example, a treatment modality for angina pectoris may be carried out according to the following sequence of procedures:

1. A stimulator 150 is implanted in the spinal column so its electrodes 156 and/or 158 are adjacent to a dorsal root 110 and/or ventral root 111.

2. Using Function 2 described above (i.e., transmitting data) of external electronic appliance 200 and external appliance 190, implantable stimulator 150 is commanded to produce a series of excitatory electrical stimulation pulses.

3. Set stimulator on/off period to an appropriate setting, e.g., continuously on.

4. After each stimulation pulse, series of pulses, or at some other predefined interval, any change in ECG suggestive of angina pectoris is sensed, preferably by one or more electrodes 156 and 158 of implantable stimulator 150. These responses are converted to data and telemetered out to external electronic appliance 200 via Function 3.

5. From the response data received at external appliance 200 from the implantable stimulator 150, or from other assessment, the stimulus threshold for obtaining a reflex response is determined and is used by a clinician acting directly 212 or by other computing means 218 to transmit the desired stimulation parameters to the implantable stimulator 150 in accordance with Function 2. Alternatively, external appliance 200 makes the proper adjustments automatically, and transmits the proper stimulation parameters to stimulator 150. In yet another alternative, stimulator 150 adjusts stimulation parameters automatically based on the sensed response.

6. When patient 170 desires to invoke an electrical stimulation to alleviate symptoms (e.g., pain, loss of function, etc.), patient 170 employs handheld controller 180 to set the implantable stimulator 150 in a state where it delivers a prescribed stimulation pattern from a predetermined range of allowable stimulation patterns.

7. Patient 170 employs controller 180 to turn off stimulator 150, if desired.

8. Periodically, the patient or caregiver recharges the power source/storage device 162 of implantable stimulator 150, if necessary, in accordance with Function 1 described above (i.e., transmit electrical power).

For the treatment of any of the various types and classes of angina and/or PVD, it may be desirable to modify or adjust the algorithmic functions performed by the implanted and/or external components, as well as surgical approaches. For example, in some situations, it may be desirable to employ more than one implantable stimulator 150, each of which could be separately controlled by means of a digital address. Multiple channels and/or multiple patterns of stimulation might thereby be programmed by the clinician and controlled by the patient in order to, for instance, deal with complex or multidimensional pain such as may occur as a result of poor circulation in multiple limbs.

In some embodiments discussed earlier, microstimulator 150, or two or more microstimulators, is controlled via closed-loop operation. A need for and/or response to stimulation is sensed via microstimulator 150, or by an additional microstimulator (which may or may not be dedicated to the sensing function), or by another implanted or external device. If necessary, the sensed information is transmitted to microstimulator 150. In some cases, the sensing and stimulating are performed by one stimulator. In some embodiments, the stimulation parameters used by microstimulator 150 are automatically adjusted based on the sensed information. For instance, one “microstimulator” may performs the sensing, stimulation parameter adjustments, and current generating functions. Thus, the stimulation parameters may be adjusted in a closed-loop manner to provide stimulation tailored to the need for and/or response to stimulation.

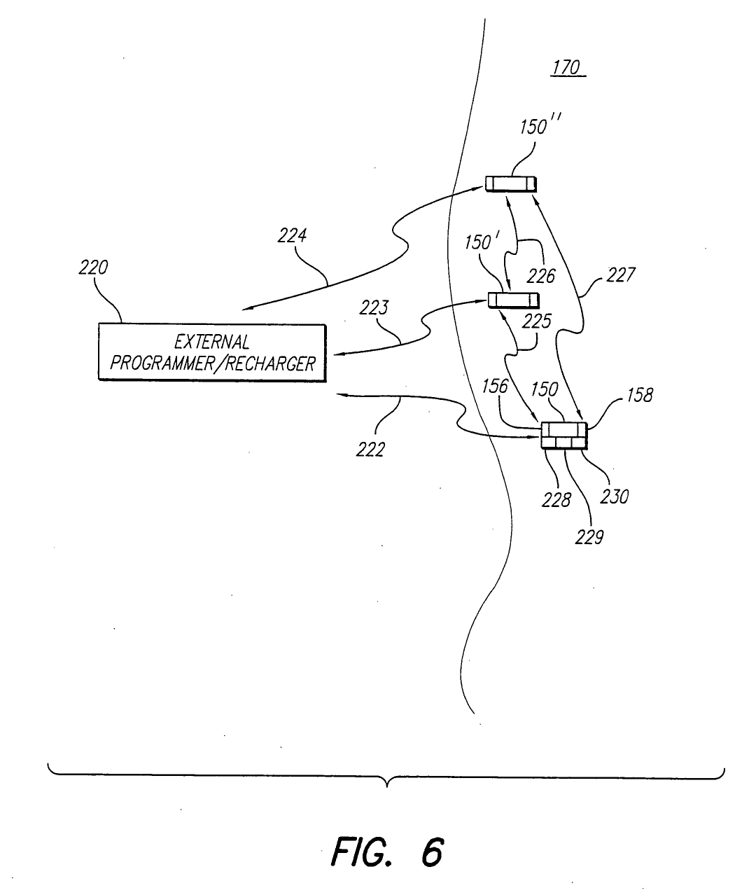

For example, as seen in FIG. 6, a first microstimulator 150, implanted in a first location, provides electrical stimulation to a first location, e.g., a left upper lumbar spinal cord dorsal column; a second microstimulator 150′ provides electrical stimulation to a second location, e.g., a right upper lumbar dorsal column; and a third microstimulator 150″ provides electrical stimulation to a third location, e.g., a left lower lumbar dorsal column. As mentioned earlier, the implanted devices may operate independently or may operate in a coordinated manner with other similar implanted devices, other implanted devices, or other devices external to the patient's body, as shown by the control lines 222, 223 and 224 in FIG. 6. That is, in accordance with certain embodiments of the invention, an external controller 220 controls the operation of one or more of the implanted microstimulators 150, 150′ and 150″.

According to various embodiments of the invention, an implanted device, e.g., microstimulator 150, may control or operate under the control of another implanted device(s), e.g., microstimulator 150′ and/or microstimulator 150″. That is, a device made in accordance with the invention may communicate with other implanted stimulators, other implanted devices, and/or devices external to a patient's body, e.g., via an RF link, an ultrasonic link, a thermal link, an optical link, or the like. Specifically, as illustrated in FIG. 6, microstimulator 150, 150′, and/or 150″, made in accordance with the invention, may communicate with an external remote control (e.g., patient and/or physician programmer 220 and/or the like) that is capable of sending commands and/or data to implanted devices and may also be capable of receiving commands and/or data from implanted devices.

Microstimulators made in accordance with the invention further incorporate, in some embodiments, first sensing means 228 for sensing therapeutic effects, clinical variables, or other indicators of the state of the patient, such as perfusion (e.g., limb perfusion) via a doppler flowmeter, or sympathetic trunk activity via ENG. The stimulators additionally or alternatively incorporate second means 229 for sensing, e.g., levels and/or changes in catecholamines and/or other markers of the potential for pain. The stimulators additionally or alternatively incorporate third means 230 for sensing electrical current levels and/or waveforms supplied by another source of electrical energy. Sensed information may then be used to control the parameters of the stimulator(s) in a closed loop manner, as shown by control lines 225, 226, and 227. Thus, the sensing means may be incorporated into a device that also includes electrical stimulation means, or the sensing means (that may or may not have stimulating means), may communicate the sensed information to another device(s) with stimulating means.

Thus, for the treatment of angina and/or the symptoms thereof, according to the present invention, the target site(s) of electrical stimulation include the cervical and/or thoracic spinal cord dorsal column(s) 120, and/or the cervical and/or thoracic spinal root(s). As shown in FIGS. 2A and 2B, dorsal columns 120 include cuneate fasciculus 122 and gracile fasciculus 124. As shown in FIGS. 2A and 3, the spinal roots lie within the spinal column, and include dorsal (posterior) root 110 and ventral (anterior) root 111. Such treatment may be targeted to increase coronary blood circulation as a means to control angina, angina pain, or other symptoms. Such a result is most likely with excitatory stimulation (applied at a relatively low frequency, e.g., less than about 100-150 Hz).

In addition, for the treatment of peripheral vascular disease (PVD) of the lower limb(s), the target site(s) of electrical stimulation include the lumbar and/or sacral spinal cord dorsal column(s) 120 (cuneate fasciculus 122 and/or gracile fasciculus 124) and/or the lumbar and/or sacral spinal root(s) (dorsal root 110 and/or ventral root 111). For treatment of peripheral vascular disease of the upper limb(s), the target site(s) of electrical stimulation include the cervical and/or thoracic spinal cord dorsal column(s) 120 (cuneate fasciculus 122 and/or gracile fasciculus 124) and/or the cervical and/or thoracic spinal root(s) (dorsal root 110 and/or ventral root 111). Such treatment may be targeted to increase peripheral blood circulation as a means to control PVD, PVD pain, or other symptoms related to PVD and its sequelae. Such a result is most likely with excitatory stimulation (applied at a relatively low frequency, e.g., less than about 100-150 Hz).

Furthermore, sensing means described earlier may be used to orchestrate first the activation of microstimulator(s) targeting one or more nerve fibers, and then, when appropriate, the microstimulator(s) targeting nerve fibers in another area and/or by a different means. Alternatively, this orchestration may be programmed, and not based on a sensed condition. In yet another alternative, this coordination may be controlled by the patient via the patient programmer.

While the invention herein disclosed has been described by means of specific embodiments and applications thereof, numerous modifications and variations could be made thereto by those skilled in the art without departing from the scope of the invention set forth in the claims.

What is claimed is:

1. A method for treating a patient with peripheral vascular disease (PVD) or angina, comprising:
providing a miniature leadless implantable stimulator with at least one electrode and with a size and shape suitable for placement entirely within the spinal column;
implanting the stimulator adjacent to at least one tissue influencing blood circulation, which tissue is at least one of the spinal roots;
providing operating power to the stimulator;
using an external appliance to transmit stimulation parameters to the stimulator;
receiving the stimulation parameters at the stimulator;
generating stimulation pulses in accordance with the stimulation parameters, which pulses are generated by the stimulator;
delivering stimulation pulses via the stimulator to the at least one of the spinal roots influencing blood circulation as a treatment for PVD or angina.

2. The method of claim 1 further comprising delivering stimulation pulses to at least one of the lumbar dorsal roots, lumbar ventral roots, sacral dorsal roots, and sacral ventral roots as a treatment for PVD of at least one lower limb.

3. The method of claim 1 further comprising delivering stimulation pulses to at least one of the cervical dorsal roots, cervical ventral roots, thoracic dorsal roots, and thoracic ventral roots as a treatment for PVD of at least one upper limb.

4. The method of claim 1 further comprising delivering excitatory stimulation pulses to increase peripheral blood circulation as a treatment for PVD.

5. The method of claim 1 further comprising delivering stimulation pulses to at least one of the cervical dorsal roots, cervical ventral roots, thoracic dorsal roots, and thoracic ventral roots as a treatment for angina.

6. The method of claim 5 further comprising delivering excitatory stimulation pulses to increase coronary blood circulation as a treatment for angina.

7. The method of claim 1 wherein the implantable stimulator further comprises at least one sensor and the method further comprises sensing at least one condition of the patient.

8. The method of claim 7 wherein the at least one sensed condition is used to adjust the stimulation parameters.

9. The method of claim 8 wherein the parameter adjustment is performed using the at least one external appliance.

10. The method of claim 8 wherein the parameter adjustment is performed by the implantable stimulator.

11. The method of claim 1 further comprising
providing at least one sensor;
using the at least one sensor to sense a physical condition; and
adjusting the stimulation parameters based on the sensed condition.

12. A method for treating a patient with peripheral vascular disease (PVD) or angina, comprising:
providing a miniature implantable stimulator with at least one electrode and with a size and shape suitable for placement of the entire stimulator within the spinal column;
implanting the stimulator adjacent to at least one tissue influencing blood circulation, which tissue is at least one of the spinal roots;
providing operating power to the stimulator;
using an external appliance to transmit stimulation parameters to the stimulator;
receiving the stimulation parameters at the stimulator;
generating stimulation pulses in accordance with the stimulation parameters, which pulses are generated by the stimulator;
delivering stimulation pulses via the stimulator to the at least one of the spinal roots influencing blood circulation as a treatment for PVD or angina.

13. The method of claim 12 further comprising delivering stimulation pulses to at least one of the lumbar dorsal roots, lumbar ventral roots, sacral dorsal roots, and sacral ventral roots as a treatment for PVD of at least one lower limb.

14. The method of claim 12 further comprising delivering stimulation pulses to at least one of the cervical dorsal roots, cervical ventral roots, thoracic dorsal roots, and thoracic ventral roots as a treatment for PVD of at least one upper limb.

15. The method of claim 12 further comprising delivering excitatory stimulation pulses to increase peripheral blood circulation as a treatment for PVD.

16. The method of claim 12 further comprising delivering stimulation pulses to at least one of the cervical dorsal roots, cervical ventral roots, thoracic dorsal roots, and thoracic ventral roots as a treatment for angina.

17. The method of claim 16 further comprising delivering excitatory stimulation pulses to increase coronary blood circulation as a treatment for angina.

18. The method of claim 12 wherein the implantable stimulator further comprises at least one sensor and the method further comprises sensing at least one condition of the patient.

19. The method of claim 18 wherein the at least one sensed condition is used to adjust the stimulation parameters.

20. The method of claim 12 further comprising
providing at least one sensor;
using the at least one sensor to sense a physical condition; and
adjusting the stimulation parameters based on the sensed condition.
